SEQUENCE LISTING (1 sequence)

SEQ ID NO: 1 - protein, 13 aa, Artificial Sequence
  Artificial Sequence
  NLS  from SV-40 large T-antigen
CGYGPKKKRKVGG

(19) United States
(12) Patent Application Publication
(10) Pub. No.: US 2020/0079858 A1
(43) Pub. Date: Mar. 12, 2020

(54) ANTIBODY CONJUGATE FOR TREATING AND DETECTING BLADDER CANCER

(71) Applicants: UNIVERSITY OF SOUTH AUSTRALIA, Adelaide (AU); CENTRAL ADELAIDE LOCAL HEALTH NETWORK INC., Adelaide (AU); SOCIETE DE COMMERCIALISATION DES PRODUITS DE LA RECHERCHE APPLIQUEE SOCPRA SOCPRA SCIENCES SANTE ET, Sherbrooke (CA)

(72) Inventors: Jeffrey Victor LEYTON, Sherbrooke (CA); Eric MARSAULT, Sherbrooke (CA); Simon BEAUDOIN, Sherbrooke (CA); Pierre-Luc BOUDREAULT, Sherbrooke (CA); Marc-André BONIN, Sherbrooke (CA); Angel LOPEZ, Medindie (AU)

(73) Assignees: UNIVERSITY OF SOUTH AUSTRALIA, Adelaide (AU); CENTRAL ADELAIDE LOCAL HEALTH NETWORK INC., Adelaide (AU); SOCIETE DE COMMERCIALISATION DES PRODUITS DE LA RECHERCHE APPLIQUEE SOCPRA SOCPRA SCIENCES SANTE ET

(21) Appl. No.: 16/493,845

(22) PCT Filed: Mar. 14, 2018
(86) PCT No.: PCT/CA2018/050303

§ 371 Date: Sep. 13, 2019

Related U.S. Application Data
(60) Provisional application No. 62/471,052, filed on Mar. 14, 2017.

(51) Int. Cl. C07K 16/28; G01N 33/574; A61K 47/54; A61K 47/68; A61K 51/04; A61K 51/10

(52) U.S. Cl. CPC C07K 16/2866; G01N 33/57407; A61K 47/554; A61K 47/6805; A61K 47/6831; A61K 47/6861; C07K 2317/21; A61K 51/1093; A61K 51/106; A61K 51/1033; C07K 2319/09; C07K 2317/20; A61K 51/0493

(57) ABSTRACT

The present description relates to a conjugated anti-interleukin-5 receptor α-subunit (IL-5Rα) compound comprising cholic acid (ChAc) or a variant thereof, the ChAc conjugated to a non-cell penetrating peptide comprising a nuclear localization sequence (NLS) conjugated to an anti-interleukin-5 receptor α-subunit (IL-5Rα) compound and further conjugated to chemotherapeutic agent and/or a radionuclide.

### CROSS-REFERENCE TO RELATED APPLICATIONS

The present application claims benefit of U.S. Provisional Application No. 62/471,052 filed Mar. 14, 2017, the content of which is hereby incorporated by reference in their entirety.

### TECHNICAL FIELD

The present relates to an antibody conjugate compound for detecting or treating muscle invasive bladder cancer.

### BACKGROUND ART

Although armed antibodies (hereinafter antibody-conjugate [AC]) delivering molecules for the imaging or treatment of targeted tissues is now a prominent approach in medicine, enhancing AC cellular retention and tumor uptake is necessary to improve their effectiveness. The increased residence time inside cancer cells and overall tumor uptake can have important implications for improved tumor killing and detection. Small molecules and radionuclides transported into cells by ACs are sensitive to a variety of mechanisms that ultimately leads to poor accumulation. Thus, new strategies for intracellular delivery technologies are needed to improve AC tumor cell accumulation and potentially effectiveness for its intended application. For example, one potential target is bladder cancer which is one of the most prevalent cancers impacting adults worldwide. Patients often have local and distant disease at the time of initial diagnosis of the primary tumor. This is significant as patients with organ confined bladder cancer undergo different treatment regimens compared to patients with metastatic disease. Thus, targeted therapeutic approaches are an active area of research to more accurately stage pre-treatment bladder cancer.

The hydrophobic interiors of cellular membranes are barriers for ACs to efficiently access the intracellular environment, which limits controlled placement and accumulation of delivered molecular payloads such as chemotherapeutics and radioisotopes. Upon receptor-mediated internalization, current ACs are reliant on entrapment inside the endosomal-lysosomal trafficking pathway where cathepsin-mediated degradation is exploited for payload release. Unfortunately, this trafficking pathway often impedes the efficient intracellular accumulation of these payloads multifold. First, ACs may undergo increased recycling, which has been shown to be a limiting factor for tumor imaging and cytotoxic effectiveness. Second, upon degradation these payloads are released near the cell surface where they are actively exported by overexpressed membrane associated transport proteins. Three, cell surface receptors may be downregulated. In addition, delivering biological payloads (i.e. toxins) that recognize intracellular targets can be degraded and rendered inactive. Thus, a technology that i) would enable ACs to efficiently escape the endosomal-lysosomal pathway, and ii) subsequently route to alternative subcellular locations could greatly enhance payload placement and accumulation and, hence, effectiveness.

Technologies such as synthetic peptides or polymers coupled to antibody surface residues enabling ACs with endosome escape or subcellular destination routing controls are known in the art. In general, these strategies exploit cellular mammalian physiology such as pH-sensitive endosome membrane-destabilizing activities or nuclear and mitochondrial-specific localization sequences. First-generation peptides were developed from a class of agents known as cell-penetrating peptides (CPPs). ¹³ Although CPP-conjugated antibodies are remarkable for their ability to ‘penetrate’ membranes and accumulate payloads with high cellular accumulation, penetration is indiscriminate. In vivo, peptide-ACs suffer from increased accumulation in non-target tissue resulting in poor tumor targeting. This is most likely caused by a change in the overall AC net charge due to modification with large cationic/anionic peptides. An increase in net charge has been shown to increase AC plasma clearance or increase distribution in normal tissues.

While short peptides capable or penetrating plasma membranes or harboring nuclear localization signal (NLS) sequences represent an excellent platform for increasing mAb conjugate cellular accumulation, it is often at the expense of hindered specificity. As monoclonal antibodies (mAbs) including next-generation humanized and fully human antibodies are well established for the treatment of cancer, their excellent target-specific affinity and specificity provide mAbs with the potential to naturally extend into the clinic as diagnostic agents.

A recent advancement in ACs functionalization has been to empower ACs to achieve multi-selective targeting by attaching peptides that harbor compartment-localizing amino acids. In particular, the nuclear localization signal (NLS) sequence from SV-40 Large T-antigen has previously been incorporated into synthetic peptides and conjugated to proteins and demonstrated the ability to direct the transport of proteins into the nucleus. Although, the optimized NLS sequence is 25 amino acids long, the mAb 7G3 was conjugated to a 13-mer peptide (CGYGPKKKRKVGG) harboring a segment of the NLS (underlined) sufficient for nuclear translocation. An advantage of this short sequence is that it does not penetrate cells and allows mAbs to maintain cell selectivity. 7G3-NLS was used to deliver the radioisotope cargo indium-111 (¹¹¹In) inside the nucleus. Molecular damage by ¹¹¹In is due to its emissions of energetic Auger electrons. Because they travel only nanometer-micrometer distances they are more effective if delivered inside the nucleus. Unfortunately, cytotoxicity was not overwhelming relative to standard ¹¹¹In-7G3 and the evidence suggested it was due to ineffective nuclear localization caused by entrapment in the endosomal-lysosomal and/or recycling pathways.

Therefore, there is still a need to be provided with an AC that can be used in detecting or treating cancer such as bladder cancer.

### SUMMARY

In accordance with the present description, there is now provided a conjugated anti-interleukin-5 receptor α-subunit (IL-5Rα) compound comprising cholic acid (ChAc) or a variant thereof, said ChAc conjugated to a non-cell penetrating peptide comprising a nuclear localization sequence (NLS) conjugated to an anti-interleukin-5 receptor α-subunit (IL-5Rα) compound.

In an embodiment, the anti-IL-5Rα compound is an antibody.

In another embodiment, the antibody is a monoclonal or polyclonal antibody.

In a further embodiment, the antibody is a mouse antibody, a goat antibody, a human antibody or a rabbit antibody.

In an embodiment, the antibody is a humanized antibody.

In another embodiment, the antibody comprises an epitope binding fragment selected from the group consisting of: Fv, F(ab′) and F(ab′)2.

In a further embodiment, the nuclear localization sequence is from SV40 large T antigen.

In another embodiment, the non-cell penetrating peptide comprises at least one spacer residue.

In an additional embodiment, the non-cell penetrating peptide comprises at least one cysteine for coupling to ChAc and the anti-IL-5Rα compound.

In a further embodiment, the non-cell penetrating peptide is as set forth in SEQ ID NO: 1.

In another embodiment, the compound of interest is an A14 antibody.

In a further embodiment, the ratio of ChAcNLS peptide conjugated per compound of interest is between 1 to 10 peptides per compound.

In another embodiment, the conjugated compound described herein further comprises a radionuclide attached thereto.

In an embodiment, the radionuclide is at least one of ⁴⁷Sc, ⁵¹Cr, ⁵²mMn, ⁵⁵Co, ⁵⁸Co, ⁵²Fe, ⁵⁶Ni, ⁵⁷Ni, ⁶¹Cu, ⁶²Cu, ⁶⁴Cu, ⁶⁷Cu, ⁶⁶Ga, ⁶⁸Ga, ⁶⁷Ga, ⁷²As, ⁷⁷As, ⁸⁹Zr, ⁹⁰Y, ⁹⁴mTc, ⁹⁹mTc, ⁹⁷Ru, ¹⁰⁵Rh, ¹⁰⁹Pd, ¹¹¹Ag, ¹¹⁰In, ¹¹¹In, ¹¹³mln, ¹¹⁴mln, ¹¹⁷mSn, ¹²¹Sn, ¹²⁷Te, ¹⁴²Pr, ¹⁴³Pr, ¹⁴⁹Pm, ¹⁵¹Pm, ¹⁴⁹Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, 172Tm, 177Lu, ¹⁸⁶Re, ¹⁸⁸Re, ¹⁹¹Pt, ¹⁹⁷Hg, ¹⁹⁸Au, ¹⁹⁹Au, ²⁰¹Tl, ²⁰³Pb, ²¹¹At, ²¹²Bi, ²¹³Bi, ¹¹C, ⁷⁵Br, ⁷⁶Br, ⁷⁷Br, ⁸²Br, ¹⁸F, ¹²⁰I, ¹²³I, ¹²⁴I, ¹²⁵I, ¹³¹I, ⁸⁹Sr and ²²⁵Ac.

In a further embodiment, the radionuclide is ⁶⁴Cu.

In another embodiment, the conjugated compound described herein further comprises a chemotherapeutic agent attached thereto.

In an embodiment, the chemotherapeutic agent is vinblastine or α-amanitin.

In an additional embodiment, the compound described herein is for treating bladder cancer.

In another embodiment, the compound described herein is for detecting bladder cancer.

In a further embodiment, the bladder cancer is muscle invasive bladder cancer (MIBC).

In another embodiment, the compound described herein is for detecting bladder cancer by PET imaging.

It is further provided herein the use of the conjugated anti-IL-5Rα compound as described herein for detecting and/or treating bladder cancer.

It is also provided the use of the conjugated anti-IL-5Rα compound as described herein in the manufacture of a medicament for treating bladder cancer.

It is also provided herein a method of detecting and/or treating bladder cancer in a subject, comprising the step of administering to a subject the conjugated anti-IL-5Rα compound as described herein.

In an embodiment, the subject is a mouse or a human.

It is also provided an anti-bladder cancer compound comprising cholic acid (ChAc) or a variant thereof, said ChAc conjugated to a non-cell penetrating peptide comprising a nuclear localization sequence (NLS) conjugated to an anti-interleukin-5 receptor α-subunit (IL-5Rα) compound, and conjugated to a chemotherapeutic agent attached thereto.

### BRIEF DESCRIPTION OF THE DRAWINGS

Reference will now be made to the accompanying drawings.

FIG. 1 illustrates a schematic representation of ChAcNLS conjugated to surface lysines via the crosslinker sulfo-SMCC.

FIG. 2 illustrates HT-1376 cells incubated with A14 or A14-ChAcNLS and showing in (A) increase in ceramide levels quantified by flow cytometric analysis and represented as fluorescence intensity relative to control at 2 h and 6 h incubations; (B) confocal microscopy images of GFP-Galectin-3-HT-1376 cells incubated for 1 h wherein colocalization A14 and GFP-Galectin-3 is shown by arrows; (C) histograms (left panels) showing the percentage of fluorescence colocalization (upper right quadrants) between Hoechst and the anti-A14 Ab-Alexa647, and right panels: representative images to visualize the colocalization between Hoechst and A14, and * indicates p<0.0001.

FIG. 3 illustrates in (A) the radiochemical purity and the amount of NLS or ChAcNLS per antibody assessed by non-reducing SDS-PAGE and autoradiography (left) and reducing SDS-PAGE (right), respectively, wherein L=protein standard ladder; and in (B) saturation binding affinity for ⁶⁴Cu-A14 and ⁶⁴Cu-A14-ChAcNLS determined on HT-1376 (left) and HT-B9 (right).

FIG. 4 illustrates HT-1376 (left panels) and HT-B9 (right panels) treated with ⁶⁴Cu-A14, ⁶⁴Cu-A14-NLS, and ⁶⁴Cu-A14-ChAcNLS, the ⁶⁴Cu intracellular, nuclear, and membrane accumulation represented as the fold-increase relative to ⁶⁴Cu-A14 at 1 h.

FIG. 5 illustrates HT-1376 (left panels) and HT-B9 (right panels) treated with ⁶⁴Cu-A14, ⁶⁴Cu-A14-ChAcNLS, and ⁶⁴Cu-IgG-ChAcNLS, the ⁶⁴Cu intracellular, nuclear, and membrane accumulation represented as the fold-increase relative to ⁶⁴Cu-A14 at 1 h.

FIG. 6 illustrates the blood radioactivity concentrations from tumor-bearing NOD/SCID mice injected with ⁶⁴Cu-A14-ChAcNLS and ⁶⁴Cu-A14 (A); and biodistribution in normal organ profile when intravenously injected in NOD/SCID mice in (B).

FIG. 7 illustrates coronal PET images at 24 h and 48 h post-injection of NOD/SCID mice bearing HT-1376 and HT-B9 tumors injected with ⁶⁴Cu-A14-ChAcNLS, ⁶⁴Cu-A14 and ⁶⁴Cu-A14-NLS, and wherein the bottom row: PET images from axial plane placed through the kidneys.

FIG. 8 illustrates in (A) ROI-calculated tumor uptake and in (B) ROI tumor/muscle ratio scores at 24 h and 48 h post-injection from NOD/SCID mice bearing HT-1376 and HT-B9 tumors intravenously injected with ⁶⁴Cu-A14-ChAcNLS, ⁶⁴Cu-A14 and ⁶⁴Cu-A14-NLS, wherein * indicates p<0.05.

FIG. 9 illustrates constructs of in (A) A14-MCC-C2-vinblastine, (B) A14-MCC-C10-vinblastine, and in (C) A14-SM(PEG)2-C2-vinblastine.

FIG. 10 illustrates A14 ADC constructs tested on IL-5Rα+ bladder cancer cells.

FIG. 11 illustrates in (A) A14-vinlblastine (MCC conjugated) modified with ChAcNLS using MCC as a crosslinker; and in (B) A14 ADC constructs including the modified A14-vinlblastine (MCC conjugated) tested on IL-5Rα+ bladder cancer cells.

### DETAILED DESCRIPTION

It is provided a novel design of compound-conjugates specific against rapidly internalizing receptors to link endosome escape and enhanced cellular uptake. More specifically, it is provided a conjugated compound comprising cholic acid conjugated to a non-cell penetrating peptide comprising a nuclear localization sequence (NLS) conjugated to the compound of interest.

It is provided a novel composite compound, cholic acid-NLS (ChAcNLS) that attaches stably to Abs. ChAcNLS utilizes biological mimicry of viruses for escaping endosome entrapment and routing to the nucleus without compromising for example Ab affinity and specificity. These independent actions are required for increasing Ab cellular accumulation.

It is provided herein in one embodiment an IL-5Rα-targeted ⁶⁴Cu-labeled mAb modified with a novel compound that improves tumor targeting and sensitive visualization of variably expressing IL-5Rα-positive muscle invasive bladder cancer (MIBC). In an embodiment, the monoclonal antibody (mAb) A14 is modified with the compound ChAcNLS that enables mAbs to escape endosome entrapment and route to the nucleus in target cells. ⁶⁴Cu-A14-ChAcNLS is able to increase cellular accumulation in target MIBC cells with high affinity and specificity. In addition, ⁶⁴Cu-A14-ChAcNLS is able to visualize tumors in IL-5Rα-positive invasive bladder tumors below the threshold of detection using standard ⁶⁴Cu-A14. The performance of ⁶⁴Cu-A14-ChAcNLS establishes a rationale for the development of mAb conjugate PET agents that take charge of their intracellular trafficking to improve tumor imaging. ⁶⁴Cu-A14-ChAcNLS also establishes an approach for IL-5Rα-targeted PET imaging. This PET tracer may impact the determination of MIBC during staging and improve therapy guidance.

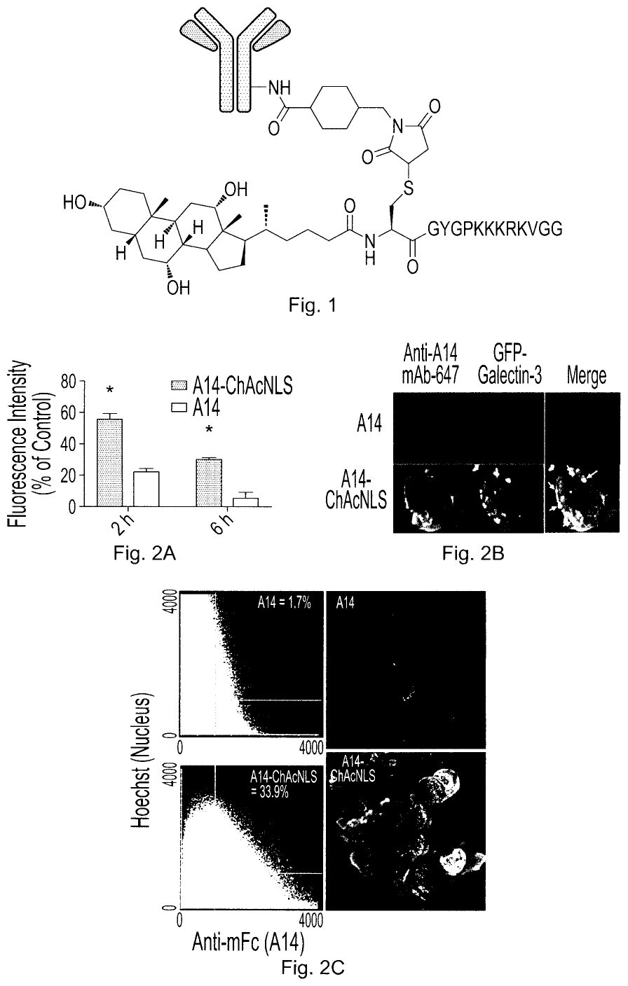

It is thus described an AC coupled with cholic acid and coupled to a 13 amino acid peptide (SEQ ID NO: 1) containing a nuclear localization sequence (FIG. 1; termed ChAcNLS) enables ACs to efficiently i) escape endosome entrapment, ii) route to the nucleus, and iii) increase intracellular accumulation of the delivered payload with high target receptor affinity and target cell selectivity in leukemia and breast cancer. Importantly, these actions increased cytotoxicity up to 100-fold when Trastuzumab-emtanisne was modified with ChAcNLS. Cholic acid and the peptide are linked via an N-terminal cysteine in a juxtapose configuration. The cysteine reacts with a maleimide on the chosen crosslinker sulfosuccinimidyl-4-(N-maleimidomethyl)cyclohexane-1-carbon/late (sulfo-SMCC) that is initially conjugated to antibody surface lysines. ChAcNLS has a molecular weight of 1.8 kDa and its ability to increase cellular accumulation and cytotoxicity is proportional to the quantity of peptide conjugated on to the AC.

In an embodiment, the antibody encompassed herein is a monoclonal or polyclonal antibody.

In another embodiment, the antibody is a mouse antibody, a goat antibody, a human antibody or a rabbit antibody, or a humanized antibody.

Also encompassed, the antibody might comprises an epitope binding fragment selected from the group consisting of: Fv, F(ab′), or F(ab′)2.

As showed herein, it is demonstrated that a ChAcNLS-modified AC has tumor targeting properties in vivo. It is also demonstrated the ability of A14-ChAcNLS to escape endosome entrapment and localize to the nucleus. PET imaging, biodistribution, and pharmacokinetic (PK) analysis were used to determine the impact on tumor and normal organ uptake, and hence, tumor targeting.

The interleukin-5 receptor α-subunit (IL-5Rα) is a target for PET imaging of muscle invasive bladder cancer (MIBC) using radiolabeled mAbs. Until recently, IL-5Rα had a limited role in cancer. However, IL-5Rα is preferentially overexpressed in invasive bladder cancer from a transcriptomic perspective (Lee et al., 2012, PLoS One, 7(9): e40267). Moreover, IL-5 treatment amplified components associated with cancer invasion such as enhanced cellular migration, expression of matrix metalloproteinases (MMPs), and the arrest of cellular proliferation. Using immunohistochemical analysis of primary bladder specimens obtained from 134 patients undergoing tumor resections or complete bladder removal, it was found that IL-5Rα protein was preferentially overexpressed in invasive bladder tumors relative to non-invasive tumors and healthy urothelial tissues. Furthermore, IL-5Rα has rapid internalization and re-expression dynamics, which were attractive for mAb targeting. To develop a potential PET imaging agent, the IL-5Rα-specific mAb A14 was conjugated to the chelator 2,2′-(7-(2-((2,5-dioxopyrrolidin-1-yl)oxy)-2-oxoethyl)-1,4,7-triazonane-1,4-diyl)diacetic acid (NOTA-NHS) for complexation to copper-64 (⁶⁴Cu; t½=12.7 h). ⁶⁴Cu-A14 efficiently accumulated radioactivity inside target human IL-5Rα-positive invasive bladder cancer cell lines HT-1376 and HT-B9, which were grown bilaterally on the flanks of NOD/SCID mice. Immunohistochemical analysis of these xenografts revealed these tumor models were similar to the IL-5Rα expression heterogeneity observed in the patient tumors. Specifically, HT-1376 xenografts were comprised of >66% of IL-5Rα-positive tumor cells. In contrast, HT-B9 tumors were comprised of only 11% IL-5Rα-positive tumor cells that were present as small ‘island’ populations. Thus, these xenografts provided tumor models with high and low tumor cell densities for assessing IL-5Rα targeting. PET imaging at 48 h post-injection and examination by region-of-interest (ROI) analysis revealed that ⁶⁴Cu-A14 can visualize HT-1376 tumors with high contrast. In comparison, ⁶⁴Cu-A14 uptake in HT-B9 tumors was difficult to visualize due to reduced accumulation and hence, PET tumor signal.

It is described herein that ChAcNLS can be applied to ⁶⁴Cu-A14 to improve its PET imaging of IL-5Rα-positive invasive bladder tumors by delivering ⁶⁴Cu via a mechanism that increases intracellular accumulation in target cancer cells. Thus, PET imaging was used in the developed IL-5Rα tumor models to determine if ⁶⁴Cu-A14-ChAcNLS improves specific tumor detection relative to ⁶⁴Cu-A14. Fluorescence and genetic methods were used to demonstrate ChAcNLS mimics Calciviridae endosome escape followed by nuclear routing, and radioimmunoassays were used to show improved ⁶⁴Cu cellular accumulation, and PET imaging and biodistribution analysis were also used to demonstrate ⁶⁴Cu-A14-ChAcNLS provides increased contrast tumor images of HT-1376 and HT-B9 tumors at 24 h post-injection compared to ⁶⁴Cu-A14. It is thus demonstrated that ⁶⁴Cu-A14-ChAcNLS outperforms ⁶⁴Cu-A14-NLS (no cholic acid) for detecting MIBC tumors.

Also encompassed herein, but not limited, are radionuclide conjugated to the compound described herein selected from ⁴⁷Sc, ⁵¹Cr, ⁵²mMn, ⁵⁵Co, ⁵⁸Co, ⁵²Fe, ⁵⁶Ni, ⁵⁷Ni, ⁶¹Cu, ⁶²Cu, ⁶⁴Cu, ⁶⁷Cu, ⁶⁶Ga, ⁶⁸Ga, ⁶⁷Ga, ⁷²As, ⁷⁷As, ⁸⁹Zr, ⁹⁰Y, ⁹⁴mTc, ⁹⁹mTc, ⁹⁷Ru, ¹⁰⁵Rh, ¹⁰⁹Pd, ¹¹¹Ag, ¹¹⁰In, ¹¹¹In, ¹¹³mln, ¹¹⁴mln, ¹¹⁷mSn, ¹²¹Sn, ¹²⁷Te, ¹⁴²Pr, ¹⁴³Pr, ¹⁴⁹Pm, ¹⁵¹Pm, ¹⁴⁹Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, 172Tm, 177Lu, ¹⁸⁶Re, ¹⁸⁸Re, ¹⁹¹Pt, ¹⁹⁷Hg, ¹⁹⁸Au, ¹⁹⁹Au, ²⁰¹Tl, ²⁰³Pb, ²¹¹At, ²¹²Bi, ²¹³Bi, ¹¹C, ⁷⁵Br, ⁷⁶Br, ⁷⁷Br, ⁸²Br, ¹⁸F, ¹²⁰I, ¹²³I, ¹²⁴I, ¹²⁵I, ¹³¹I, ⁸⁹Sr and ²²⁵Ac. In addition, also encompassed are a chemotherapeutic conjugated as described herein, as for example vinblastine.

Accordingly, it was mechanistically validated that ChAcNLS enables A14 to escape endosome entrapment and localize to the nucleus in MIBC cells. A14-ChAcNLS treated HT-1376 cells contained increased intracellular antibody levels relative to cells treated with A14. Quantifying the overlap in fluorescence between the nuclear Hoechst stain and A14 determined a 33.9% colocalization for A14-ChAcNLS (FIG. 2). In contrast, there was only 1.7% fluorescence colocalization in cells treated with A14.

Enhanced nuclear and cellular accumulation coincided with events observed with Calciviridae liberation into the cytoplasm, namely the increased production of ceramide and intracellular vesicle disruption. A14-ChAcNLS treatment increased the amount of ceramide present by a factor of ˜3 (p<0.001) at 2 h over standard treatment with A14. The increased amount of ceramide remained increase by a factor of >2 at 6 h. By 16 h ceramide levels from cells treated with A14-ChAcNLS returned to levels observed from cells treated with A14. When HT-1376 cells transfected with GFP-Galectin-3, a marker for endosomal disruption, were treated with A14-ChAcNLS there were significant (p<0.05) increases in the number of intracellular fluorescent foci from 15 to 240 min. The foci per cell response in HT-1376 cells treated with A14-ChAcNLS was >4 times higher than in the control with a peak increase of ˜16-times higher at 60 min. There was also an increased amount of fluorescence specific for A14-ChAcNLS in the nucleus and cytoplasm relative to A14 in GFP-Galectin-3 transfected cells.

In virology cholic acid has been shown as an essential host element exploited by viruses to escape endosome entrapment. The increase in ceramide in endosomal membranes causes the formation of channels or lipid flip-flop sufficient for proteins to cross. Calciviridae family requires the presence of cholic acid for the activation of acid sphingomyelinase (ASM), which cleaves sphingomyelin to produce ceramide. This instability was the mechanism used by these viruses to escape endosome entrapment. However, it is not yet known how cholic acid engages with the viral particle. The peptide component of ChAcNLS harbors an optimized nuclear localization sequence derived from the simian virus (SV)-40 large T-antigen, and previously shown to translocate ACs into the nucleus.

HT-1376 cells incubated with A14-ChAcNLS contained a 55.4%±4.1% increase (p<0.0001) of ceramide at 2 h over control (FIG. 2A). In contrast, ceramide levels were increased by 21.9%±2.5% with unmodified A14 over control. Ceramide levels remained significantly (p<0.0001) increased by 29.9%±1.4% in cells incubated with A14-ChAcNLS at 6 h. In contrast, ceramide levels were increased by 7.1%±1.0% in cells incubated with unmodified A14. By 16 h ceramide levels from cells incubated with A14-ChAcNLS reduced to levels observed from cells incubated with A14.

A second measure of endosome escape was to use a GFP-Galectin-3 assay. Galectin-3 is a lectin protein present diffusely throughout the cell cytosol that binds the β-galactoside sugars on the inner leaflet of endosomes and lysosomes. 35 When these vesicles are disrupted, the β-galactoside sugars are exposed to the cytosol allowing the cytosolic Galectin-3 to bind and aggregate on the inner leaflet. There were significant (p<0.0001) increases in the number of foci per cell by factors of >3-, 5-, 10-, 5-, and 5-fold at incubation time points of 15 min, 30 min, 1 h, 2 h, and 4 h, respectively. Moreover, there was visual colocalization between A14 and GFP-Galectin-3 in cells incubated with A14-ChAcNLS but not with A14 (FIG. 2B). As anticipated, the A14-specific fluorescence intensity was much brighter in cells incubated with A14-ChAcNLS compared to unmodified A14.

Nuclear localization by staining cells for A14 and Hoechst was evaluated and using confocal microscopy to visualize and determine the percentage of colocalized fluorescence. HT-1376 cells incubated with A14-ChAcNLS contained 33.9% colocalization (FIG. 2C). In contrast, there was only 1.7% colocalization in cells incubated with A14. In cells incubated with A14-ChAcNLS, representative images show that all cell nuclei contain the presence of colocalization. In addition, a proportion of cells the colocalization occupies the entire nucleus. In contrast, images from cells incubated with unmodified A14 show only scant colocalization in the cytoplasm and not in the nucleus.

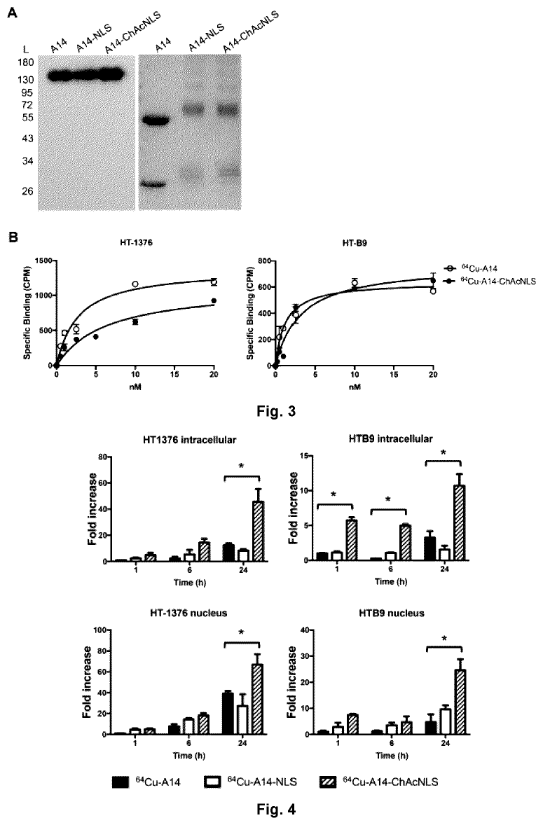

The purity of ⁶⁴Cu-A14, ⁶⁴Cu-A14-NLS, and ⁶⁴Cu-A14-ChAcNLS were A5% and ≥9% by ITLC and SDS-PAGE, respectively (FIG. 3A). The calculated retention factor (Rf) values based on a reducing 12% polyacrylamide gel, it was determined A14-NLS and A14-ChAcNLS both contained 20±2 moles of NLS and ChAcNLS per mole of antibody, respectively (FIG. 3A). The radiolabeled conjugate specific activities ranged from 220-310 MBq/mg and remained stable in PBS as <10% of the ⁶⁴Cu dissociated at 72 h after radiolabeling.

⁶⁴Cu-A14-ChAcNLS showed nanomolar affinity for IL-5Rα. A14-ChAcNLS as a function of increasing concentrations of ⁶⁴Cu-A14-ChAcNLS revealed specific binding approached saturation at concentrations of 3-5 nM in both HT-1376 and HT-B9 cells (FIG. 3B). The dissociation constant (Kd) for ⁶⁴Cu-A14-ChAcNLS on HT-1376 and HT-B9 cells was 6.4±1.7 nM and 3.1±0.8 nM, respectively. The Kd for ⁶⁴Cu-A14 on HT-1376 and HT-B9 cells was 2.7±0.6 nM and 1.2±0.3 nM, respectively.

To establish ChAcNLS-mediated increase in cellular accumulation of A14 also increases the accumulation of ⁶⁴Cu, HT-1376 and HT-B9 cell uptake experiments were performed followed by cell fractionation to dissect individual cellular subcompartments. ⁶⁴Cu-A14-ChAcNLS increased total intracellular ⁶⁴Cu accumulation compared to ⁶⁴Cu-A14 and ⁶⁴Cu-A14-NLS. In HT-1376 cells, ⁶⁴Cu-A14-ChAcNLS increased intracellular ⁶⁴Cu accumulation by factors of 9.4 and 3.2 over ⁶⁴Cu-A14 and ⁶⁴Cu-A14-NLS, respectively, at 24 h (p<0.0001; FIG. 4). In HT-B9 cells, ⁶⁴Cu-A14-ChAcNLS increased intracellular ⁶⁴Cu accumulation by factors >3.3 and >4.6 compared to ⁶⁴Cu-A14 and ⁶⁴Cu-A14-NLS, respectively, at all-time points tested (p<0.0001; FIG. 4). Nuclear radioactivity was also increased by factors of 1.7 and 2.5 at 24 h over ⁶⁴Cu-A14 and ⁶⁴Cu-A14-NLS, respectively, in HT-1376 cells. In HT-B9 cells, nuclear radioactivity for ⁶⁴Cu-A14-ChAcNLS was increased by factors of 5.3 and 2.6 relative to ⁶⁴Cu-A14 and ⁶⁴Cu-A14-NLS, respectively.

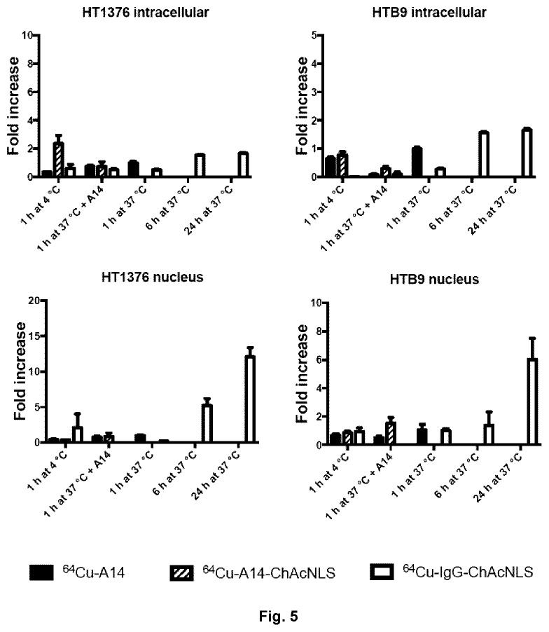

⁶⁴Cu-IgG-ChAcNLS had significantly (p<0.01) reduced nuclear and intracellular ⁶⁴Cu accumulation relative to ⁶⁴Cu-A14-ChAcNLS. (FIG. 5). There was only a scarce amount of non-specific ⁶⁴Cu accumulation in cells treated with ⁶⁴Cu-A14-ChAcNLS at 4° C. or at 37° C. plus unlabeled A14 (FIG. 5). In HT-1376 cells treated with ⁶⁴Cu-IgG-ChAcNLS and evaluated at 1 h, 6 h, and 24 h the level of intracellular ⁶⁴Cu was decreased by a factor ≥9.2 compared to ⁶⁴Cu-A14-ChAcNLS. Similarly, the level of intracellular ⁶⁴Cu was decreased by a factor compared in HT-B9 cells treated with ⁶⁴Cu-IgG-ChAcNLS compared to ⁶⁴Cu-A14-ChAcNLS. In addition, evaluating nuclear accumulation revealed the level of ⁶⁴Cu was not increased when cells were treated with ⁶⁴Cu-A14-ChAcNLS at 4° C. or at 37° C. mixed with unlabeled A14. Cells treated with ⁶⁴Cu-IgG-ChAcNLS also had reduced nuclear ⁶⁴Cu accumulation relative to ⁶⁴Cu-A14-ChAcNLS at all evaluated time points (FIG. 5). Thus, delivering ⁶⁴Cu via A14-ChAcNLS in HT-1376 and HT-B9 MIBC cells was highly effective for increasing nuclear and total cellular accumulation retaining high IL-5Rα selectivity and was internalized through a receptor-mediated process.

It has been previously shown that peptide modification of ACs correlates with increased in vitro accumulation, but can also adversely affect PK and, hence, tumor targeting properties. For example, ACs functionalized with the cationic transcriptional activator protein (TAT) peptide from human immunodeficiency virus 1 have previously been investigated as tumor targeting agents. When radiolabeled TAT-single-chain Fv antibody fragments specific for the ED-B domain of fibronectin were injected into mice, biodistribution revealed tumor uptake was reduced nearly 3-fold. The TAT-modified AC was sequestered in the spleen and liver, which was due to an 80% uptake reduction in the blood. An intact IgG-TAT suffered from similar problems resulting in substandard tumor uptake.

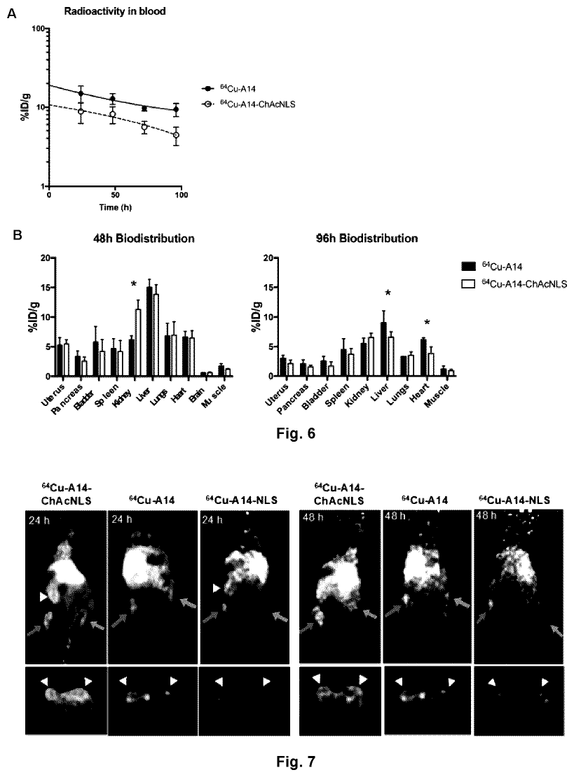

To show the impact of ChAcNLS on the blood clearance profile of ⁶⁴Cu-A14, blood sampling from 24 h to 96 h post injection was performed followed by liquid scintillation counting. Uptake was expressed in percent injected-dose/gram (% ID/g). The % ID/g in the blood from tumor bearing non-obese diabetic/severe combined immunodeficiency (NOD/SCID) mice injected with ⁶⁴Cu-A14-ChAcNLS was reduced by approximately 40% relative to 64Cu-A14 at 24 h and 48 h (p<0.001; FIG. 6A). At 72 h and 96 h, the % ID/g in the blood from mice injected with ⁶⁴Cu-A14-ChAcNLS was reduced 47% and 53% (p<0.001), respectively. The blood % ID/g reduction for both conjugates was gradual as there were no significant reductions between succeeding time points.

When the behavior of a drug in the body is known in terms of transport and distribution in tissues, a validated PK model can be used. For novel drugs such as ChAcNLS-modified ACs, a non-compartmental model is more appropriate. The estimated half-life for ⁶⁴Cu-A14-ChAcNLS was reduced by 45% (t_{1/2}=54.4 h) relative to ⁶⁴Cu-A14 (t_{1/2}=99.5 h). These data show that there is also increased blood clearance ⁶⁴Cu-A14-ChAcNLS relative to ⁶⁴Cu-A14.

Even with decreased amounts in the blood, remarkably, ⁶⁴Cu-A14-ChAcNLS is able to have comparable uptake relative to ⁶⁴Cu-A14. In HT-1376 xenografts containing >66% of IL-5Rα-positive bladder tumor cells with strong and moderate expression, the 48 h tumor uptake for ⁶⁴Cu-A14-ChAcNLS and ⁶⁴Cu-A14 was 8.0±1.0% ID/g and 8.5%±1.0% ID/g, respectively. At 96 h the tumor uptakes for ⁶⁴Cu-A14-ChAcNLS and ⁶⁴Cu-A14 were 6.0%±0.4% ID/g and 6.9%±0.3% ID/g, respectively. Unlike the HT-1376 tumors, HT-B9 tumors are comprised of only ˜11% IL-5Rα-positive MIBC cells. The tumor uptake at 48 h for ⁶⁴Cu-A14 and ⁶⁴Cu-A14-ChAcNLS was 7.2%±2.8% ID/g and 6.9%±1.8% ID/g, respectively. At 96 h the tumor uptakes for 64Cu-A14-ChAcNLS and ⁶⁴Cu-A14 were 3.2%±2.2% ID/g and 3.3%±1.2% ID/g, respectively. Thus, unlike previous peptide-modified ACs, ChAcNLS maintained good tumor uptake.

Distribution in normal organs revealed elevated kidney uptake with ⁶⁴Cu-A14-ChAcNLS at 48 h (FIG. 6B). However, at 96 h the uptake in the kidneys decreased to a comparable level observed with ⁶⁴Cu-A14. Interestingly, the bladder uptake for ⁶⁴Cu-A14-ChAcNLS was not proportionally elevated. Although not significant except for the liver and the heart, the % ID/g values were reduced in mice injected with ⁶⁴Cu-A14-ChAcNLS. However, the biodistribution data did result in multiple tumor/normal tissue ratios for ⁶⁴Cu-A14-ChAcNLS that were significantly increased relative to ⁶⁴Cu-A14 for both HT-1376 and HT-B9 tumors at both 48 h and 96 h (Tables 1 and 2). These biodistribution and PK analyses reveal that ⁶⁴Cu-A14-ChAcNLS ability to have rapid clearance from healthy tissues in combination with comparable tumor accumulation resulted in a superior tumor targeting efficiency in these IL-5Rα-positive models of MIBC.

TABLE 1
HT-1376 tumor-to-non-target tissue ratios

| | 48 Hours | | | 96 Hours | | |
|---|---|---|---|---|---|---|
| | | ⁶⁴Cu-A14- | | | ⁶⁴Cu-A14- | |
| Organs | ⁶⁴Cu-A14 | ChAcNLS | p-value* | ⁶⁴CuA14 | ChAcNLS | p-value* |
| Blood | 0.7 | 1.0 | ns | 0.7 | 1.4 | <0.0001 |
| Uterus | 1.6 | 1.5 | ns | 2.3 | 2.8 | 0.0079 |
| Pancreas | 2.5 | 3.1 | 0.0041 | 3.3 | 3.9 | 0.0008 |
| Bladder | 1.5 | 1.9 | 0.0447 | 2.7 | 3.5 | <0.0001 |
| Spleen | 1.8 | 1.9 | ns | 1.6 | 1.6 | ns |
| Kidney | 1.4 | 0.7 | 0.0001^{†} | 1.3 | 0.9 | ns |
| Liver | 0.6 | 0.6 | ns | 0.8 | 0.9 | ns |
| Lungs | 1.2 | 1.2 | ns | 2.1 | 1.7 | ns |
| Heart | 1.3 | 1.2 | ns | 1.1 | 1.6 | 0.0079 |
| Brain | 15.0 | 12.7 | <0.0001 | nd | nd | — |
| Muscle | 4.9 | 6.7 | <0.0001 | 5.9 | 6.8 | <0.0001 |

*Significance in favor of ⁶⁴Cu-A14-ChAcNLS
^{†}Significance in favor of ⁶⁴Cu-A14
ns: Not significant
nd: Not determined

TABLE 2
HT-B9 tumor-to-non-target tissue ratios

| | 48 Hours | | | 96 Hours | | |
|---|---|---|---|---|---|---|
| | | ⁶⁴Cu-A14- | | | ⁶⁴Cu-A14- | |
| Organs | ⁶⁴Cu-A14 | ChAcNLS | p-value* | ⁶⁴CuA14 | ChAcNLS | p-value* |
| Blood | 0.6 | 0.9 | ns | 0.3 | 0.8 | 0.0420 |
| Uterus | 1.4 | 1.3 | ns | 1.0 | 1.6 | 0.0050 |
| Pancreas | 2.1 | 2.7 | 0.0052 | 1.5 | 2.2 | 0.0003 |
| Bladder | 1.2 | 1.6 | 0.0447 | 1.2 | 2.0 | <0.0001 |
| Spleen | 1.5 | 1.7 | ns | 0.7 | 0.9 | ns |
| Kidney | 1.2 | 0.6 | 0.0026^{†} | 0.6 | 0.5 | ns |
| Liver | 0.5 | 0.5 | ns | 0.4 | 0.5 | ns |
| Lungs | 1.1 | 1.0 | ns | 1.0 | 1.0 | ns |
| Heart | 1.1 | 1.1 | ns | 0.5 | 0.9 | ns |
| Brain | 12.6 | 11.0 | <0.0001 | nd | nd | — |
| Muscle | 4.1 | 5.8 | <0.0001 | 2.7 | 3.8 | <0.0001 |

*Significance in favor of ⁶⁴Cu-A14-ChAcNLS
^{†}Significance in favor of ⁶⁴Cu-A14
ns: Not significant
nd: Not determined

To further evaluate tumor targeting performance, PET was performed and tissue distribution visualized. ⁶⁴Cu-A14-ChAcNLS-mediated tumor imaging was highly specific as tumor uptake in both HT-1376 and HT-B9 tumors was reduced ˜2-fold upon pre-injection of excess unlabeled A14 (p<0.05). PET images at 24 h and 48 h in mice injected with ⁶⁴Cu-A14-ChAcNLS without predosing of unlabeled A14 revealed strong uptake in HT-1376 tumors (FIG. 7). The uptake in the liver indicated hepatic clearance as the major metabolic pathway. Interestingly, there was clear annular-shaped uptake in the kidneys in mice injected with ⁶⁴Cu-A14-ChAcNLS at 24 h but that was noticeably reduced by 48 h. The annular-shaped uptake is indicative of glomerular accumulation and supports the biodistribution data that showed kidney uptake was elevated at 24 h but by 96 h the uptake decreased to levels comparable with ⁶⁴Cu-A14. In addition, there was no uptake visualized in the bladder. Biodistribution studies also showed that the bladder uptake in mice injected with ⁶⁴Cu-A14-ChAcNLS and ⁶⁴Cu-A14 were comparable. Hence, this data indicates that glomerular accumulation of ⁶⁴Cu-A14-ChAcNLS does indeed occur but is an early and transient property.

In comparison, the PET images in mice injected with ⁶⁴Cu-A14 and ⁶⁴Cu-A14-NLS revealed reduced uptake in HT-1376 tumors relative to 64Cu-A14-ChAcNLS (FIG. 7). For ⁶⁴Cu-A14, tumor uptake appeared less intense due to increased background radioactivity. In contrast, images from mice injected with ⁶⁴Cu-A14-NLS showed much less background radioactivity. Unfortunately, the tumor uptake was also noticeably reduced. The liver appeared to be the major metabolic pathway for ⁶⁴Cu-A14 and ⁶⁴Cu-A14-NLS. However, there was kidney uptake in mice injected with ⁶⁴Cu-A14-NLS.

For the HT-B9 tumors, only ⁶⁴Cu-A14-ChAcNLS was able to provide PET images with sufficient tumor-contrast (FIG. 7). HT-B9 tumors in mice injected with ⁶⁴Cu-A14-ChAcNLS had uptake that had increased focal intensity compared to mice injected with ⁶⁴Cu-A14 at 24 h and 48 h. In mice injected with ⁶⁴Cu-A14-NLS, HT-B9 tumors were unable to be visualized. Thus, ⁶⁴Cu-A14-ChAcNLS superior tumor contrast images by PET for IL-5Rα-positive MIBC tumors. In addition, for the difficult to target HT-B9 tumor, ChAcNLS modification was required to provide high contrast images.

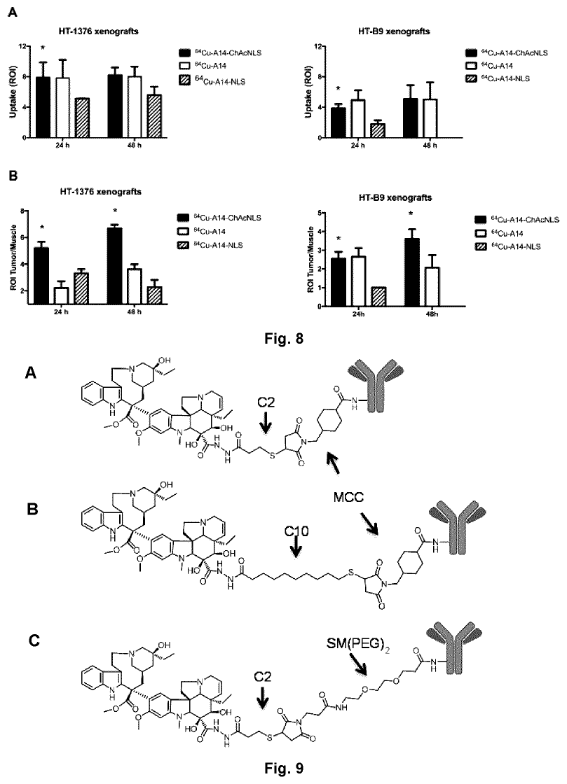

ROI analysis was used to quantify uptake in tumors and the adjacent muscle tissue to determine tumor/muscle ratios. HT-1376 tumor uptake for ⁶⁴Cu-A14-ChAcNLS was comparable with ⁶⁴Cu-A14 and was increased by 35% (p<0.05) relative to ⁶⁴Cu-A14-NLS at 24 h (FIG. 8A). This resulted in ⁶⁴Cu-A14-ChAcNLS producing tumor/muscle ratios that were increased by factors of 2.4 and 1.6 (p<0.0001) over ⁶⁴Cu-A14 and ⁶⁴Cu-A14-NLS, respectively (FIG. 8B). At 48 h, ⁶⁴Cu-A14-ChAcNLS continued with comparable tumor uptake relative to ⁶⁴Cu-A14. Although not significant, tumor uptake for ⁶⁴Cu-A14-ChAcNLS was increased by 32% over ⁶⁴Cu-A14-NLS. This resulted in ⁶⁴Cu-A14-ChAcNLS producing tumor/muscle ratios that were increased by factors of 1.8 and 2.9 (p<0.0001) over ⁶⁴Cu-A14 and ⁶⁴Cu-A14-NLS, respectively (FIG. 8B).

For HT-B9 tumors, ⁶⁴Cu-A14-ChAcNLS had comparable tumor uptake to ⁶⁴Cu-A14 and was significantly (p≤0.01) increased relative to ⁶⁴Cu-A14-NLS (FIG. 8A). At 48 h the comparable tumor uptake between ⁶⁴Cu-A14-ChAcNLS and ⁶⁴Cu-A14 continued. Tumor uptake could not be determined in mice injected with ⁶⁴Cu-A14-NLS due to the lack of visualization. ROI contrast ratio scores revealed significantly that ⁶⁴Cu-A14-ChAcNLS increased the tumor/muscle ratio over ⁶⁴Cu-A14-NLS at 24 h and ⁶⁴Cu-A14 at 48 h by factors of 2.6 and 1.7 (p<0.0001), respectively.

In the preclinical model of human IL-5Rα-positive MIBC, it is demonstrated that ⁶⁴Cu-A14-ChAcNLS is able to increase the cellular accumulation of ⁶⁴Cu via a mechanism that incorporates escape from the endosomal-lysosomal intracellular trafficking pathway coupled to nuclear localization. In vivo, ⁶⁴Cu-A14-ChAcNLS achieved increased specific exposure to MIBC tumors at equal doses versus ⁶⁴Cu-A14 and resulted in improved targeting of IL-5Rα-positive expressing MIBC tumors with high and low tumor cell densities. Importantly, this study reveals that the combination of increased cellular accumulation, faster clearance from the blood, and good tumor uptake shows that more payload can be delivered to a tumor cell per individual binding event. Thus, ChAcNLS is an effective conjugation moiety to improve AC tumor targeting. Moreover, ChAcNLS is a solution for ACs modified with peptides to deliver payloads to specific locations in the cell interior such as the nucleus. ChAcNLS did not provide increased tumor targeting for ⁶⁴Cu-A14 through extremely rapid washout, which proportionally decreases tumor uptake as occurs for other peptide-modified ACs. Instead, ⁶⁴Cu-A14-ChAcNLS displayed intermediate clearance and maintained high tumor uptake.

It is further encompassed herein the possibility of not only conjugated an antibody as described herein but also conjugating the antibody with a further drug, such as vinblastine and/or α-amanitin, which is used in combination with other chemotherapy drugs to treat bladder cancer.

Vinblastine is chemical analogue of vincristine. It binds tubulin, inhibiting the assembly of microtubules. Vinblastine is reported to be an effective component of certain chemotherapy regimens, particularly when used with bleomycin, and methotrexate, to treat a number of types of cancer, including Hodgkin's lymphoma, non-small cell lung cancer, bladder cancer, brain cancer, melanoma, and testicular cancer.

α-Amanitinis a cyclic peptide of eight amino acids, consisting of a selective inhibitor of RNA polymerase II and III and as showed a high antitumoral activity.

Accordingly, the conjugated compound described herein can be used for detecting or treating bladder cancer.

Accordingly, it is encompassed herein that the antibody-drug conjugates (ADCs) as described herein comprises a small molecule toxin such as for example and not limited to, microtubule disrupting agents (such as vinblastine, Monomethyl auristatin E or MMAE, DM1) and/or DNA alkylating agents.

For example, an antibody conjugated with ChAcNLS together with an attached chemotherapeutic molecule such as 4,4-difluoro-8-(4-carboxphenyl)-1,3,5,7-tetramethyl-4-bora-3a,4a-diaza-s-indacene (BODIPY for short), which is a cytotoxic molecule used in photodynamic therapy applications in cancer, is encompassed.

For example, A14 is attached to vinblastine via its carbon-16 position, which has previously been attached to monoclonal antibodies (mAbs) for the development of ADCs. Encompassed herein is a formulation wherein for example A14-vinblastine is constructed using the crosslinkers sulfosuccinimidyl-4-N-malimidomethylcyclohexane-1-carboxylate (MCC) and the polyethylene glycol maleimide-containing spacer (SM(PEG)2).

As described herein, A14-vinblastine conjugates were designed to include carbon chains consisting of 2 or 10 carbons spaced between the MCC/SM(PEG)2 linker and vinblastine (see FIG. 9).

The following formulations were developed and tested for cytotoxicity against IL-5Rx-positive HT-1376 MIBC cells.

TABLE 3
A14-vinblastine conjugates formulations

| Crosslinker | Ratio (crosslinker-to-A14) |
|---|---|
| C2-MCC | 5-to-1 |
| C2-MCC | 10-to-1 |
| C2-SM(PEG)₂ | 10-to-1 |
| C2-SM(PEG)₂ | 25-to-1 |
| C2-SM(PEG)₂ | 50-to-1 |
| C2-SM(PEG)₂ | 100-to-1 |
| C10-MCC | 5-to-1 |
| C10-MCC | 10-to-1 |

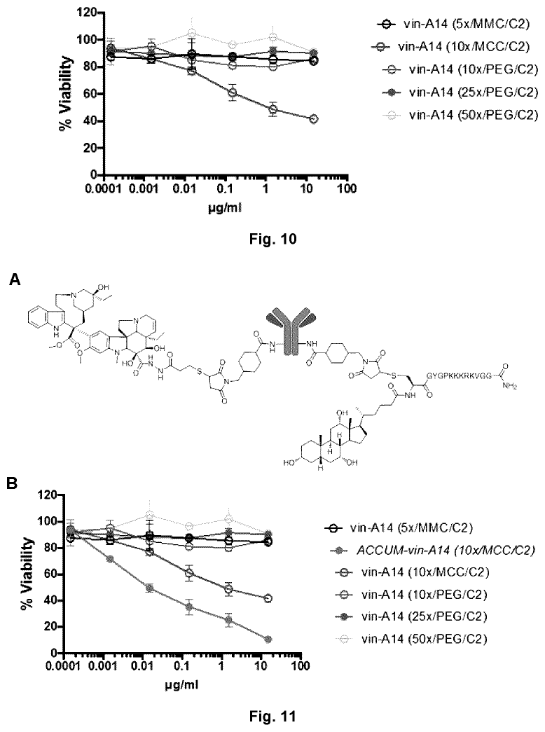

As seen in FIG. 10, the A14-vinblastine modified with MCC at a 10-to-1 MCC-to-antibody ratio displayed the most effective cytotoxicity capabilities.

It is further described that the A14-vinlblastine (MCC conjugated) as encompassed herein was modified with ChAcNLS again using MCC as a crosslinker (see FIG. 11A).

As seen in FIG. 11B, ChAcNLS-A14-vinblastine was more cytotoxic relative to A14-vinblastine by 1-log. Accordingly, as described herein, the ADC developed herein modified with ChAcNLS when tested in IL-5Rα for MIBC and when has enhanced cytotoxicity.

The present disclosure will be more readily understood by referring to the following examples which are given to illustrate embodiments rather than to limit its scope.

### Example I

### ChAcNLS Attachment and Preparation of Conjugates

A14 was obtained and purified as previously described (Sun et al., 1996, Blood, 87: 83-92.

To evaluate conjugation, a reducing 12% polyacrylamide gel was loaded with A14, A14-NLS, A14-ChAcNLS, and protein standards (BioRad, Ontario, Canada). The gel was stained with Coomassie and protein standard retention factor (Rf) values obtained. The size of the A14-NLS and A14-ChAcNLS heavy and light chains were extrapolated by plotting the distance of migration against the Rf values. The numbers of ChAcNLS or NLS compounds per A14 were calculated by dividing the difference in molecular weight (MW) between the modified A14 conjugates and unmodified A14 by 1768.5 g/mol and 1418.8 g/mol, respectively.

A14 was first reacted with NOTA-NHS in a 5-to-1 NOTA-to-A14 ratio, purified, and then conjugated to NLS or ChAcNLS followed by purification. Radiolabeling efficiency was determined by autoradiography on both instant thin-layer chromatography strips (realized in 0.1 M sodium citrate, pH 5.5) and on a non-reducing 12% polyacrylamide gel was loaded with ⁶⁴Cu-labeled conjugates (realized by SDS-PAGE).

### Example II

### Mechanistic Studies

HT-1376 cells were treated with 200 nmol/L of A14 or A14-ChAcNLS for 1 h at 37° C. followed by washing in ice cold PBS. Cells were then replenished with fresh antibody-free media and placed back at 37° C. for 1 h. Cells were then prepped for nuclear and antibody staining for evaluation by confocal microscopy as previously described (Beaudoin et al., 2016, Mol Pharm, 13: 1915-1926). For determination of ceramide levels, HT-1376 cells were treated with A14 or A14-ChAcNLS for increasing time points at 37° C. Cells were fixed and permeabilized. Cells were incubated with the anti-ceramide antibody conjugated to the fluorophore AlexaFluor 488 (Cedarlane, Ontario, Canada). Flow cytometric analysis measured the mean fluorescence intensity (MFI).

To explore endosome escape, HT-1376 cells were transfected with cDNA encoding for GFP-Galectin-3. Cells were then treated with 200 nM of A14 or A14-ChAcNLS for 1 h at 37° C. Cells were fixed and permeabilized as described above. Cells were evaluated by confocal microscopy evaluating GFP- and A14 (probed with anti-mFc AlexaFluor 647)-specific fluorescence.

### Example III

### In Vivo IL-5Rα-Positive Invasive Bladder Cancer Tumor Targeting

When tumors were 65-100 mm³, mice (n=5) were intravenously injected with ⁶⁴Cu-A14, ⁶⁴Cu-A14-NLS, or ⁶⁴Cu-A14-ChAcNLS (˜25 μg; ˜7 MBq; radiochemical purity ≥98%). Nicking of the saphenous vein and collection of blood was performed daily. Mice anesthetized under 2.5% isoflurane were then euthanized by CO₂ inhalation at 48 h and 96 h post-injection. Major organs and tumors were excised, rinsed in saline, blotted dry, and placed in pre-weighted tubes and gamma counted. Radioactivity accumulation was corrected for decay and expressed as the injected dose per gram of tissue (% ID/g).

PET imaging studies were performed on 5 mice per group on a PET/CT Triumph™ scanner (Trifoil, Calif., USA) at 24 h and 48 h post-injection. PET scans were acquired for 30 and 45 min at 24 h and 48 h, respectively, with tumors near the center of the field of view, in double axial sampling mode to improve spatial resolution. The images were reconstructed using 20 iterations of an MLEM algorithm implementing a physical description of the detectors in the system matrix. A cylindrical phantom (24.8 ml) containing 5MBq of ⁶⁴Cu at day 0 was used to obtain a calibration factor to convert the counts per seconds into kBq/mL, from which % ID/g values were derived from ROI drawings (n≥3) of the tumor, muscle, and heart using the AMIDE software.

While the present disclosure has been described in connection with specific embodiments thereof, it will be understood that it is capable of further modifications and this application is intended to cover any variations, uses, or adaptations of the including such departures from the present disclosure as come within known or customary practice within the art and as may be applied to the essential features hereinbefore set forth, and as follows in the scope of the appended claims.

1. A conjugated anti-interleukin-5 receptor α-subunit (IL-5Rα) compound comprising cholic acid (ChAc) or a variant thereof, said ChAc conjugated to a non-cell penetrating peptide comprising a nuclear localization sequence (NLS) conjugated to an anti-interleukin-5 receptor α-subunit (IL-5Rα) compound.

2. The conjugated anti-IL-5Rα compound of claim 1, wherein the anti-IL-5Rα compound is an antibody.

3. The conjugated anti-IL-5Rα compound of claim 2, wherein the antibody is a monoclonal or polyclonal antibody.

4. The conjugated anti-IL-5Rα compound of claim 2, wherein the antibody is a mouse antibody, a goat antibody, a human antibody or a rabbit antibody.

5-6. (canceled)

7. The conjugated anti-IL-5Rα compound of claim 1, wherein the nuclear localization sequence is from SV40 large T antigen.

8. The conjugated anti-IL-5Rα compound of claim 1, wherein the non-cell penetrating peptide comprises at least one spacer residue.

9. The conjugated anti-IL-5Rα compound of claim 1, wherein the non-cell penetrating peptide comprises at least one cysteine for coupling to ChAc and the anti-IL-5Rα compound.

10. The conjugated anti-IL-5Rα compound of claim 1, wherein the non-cell penetrating peptide is set forth in SEQ ID NO:1.

11. The conjugated anti-IL-5Rα compound of claim 1, wherein the anti-IL-5Rα compound is an A14 antibody.

12. The conjugated anti-IL-5Rα compound of claim 1, wherein the ratio of ChAcNLS peptide conjugated per compound of interest is between 1 to 10 peptides per compound.

13. The conjugated anti-IL-5Rα compound of claim 1, further comprising a radionuclide attached thereto.

14. The conjugated anti-IL-5Rα compound of claim 13, wherein the radionuclide is at least one of ⁴⁷Sc, ⁵¹Cr, ⁵²mMn, ⁵⁵Co, ⁵⁸Co, ⁵²Fe, ⁵⁶Ni, ⁵⁷Ni, ⁶¹Cu, ⁶²Cu, ⁶⁴Cu, ⁶⁷Cu, ⁶⁶Ga, ⁶⁸Ga, ⁶⁷Ga ⁷²As, ⁷⁷As, ⁸⁹Zr, ⁹⁰Y, ⁹⁴mTc, ⁹⁹mTc, ⁹⁷Ru, ¹⁰⁵Rh, ¹⁰⁹Pd, ¹¹¹Ag, ¹¹⁰In, ¹¹¹In, ¹¹³mln, ¹¹⁴mln, ¹¹⁷mSn, ¹²¹Sn, ¹²⁷Te, ¹⁴²Pr, ¹⁴³Pr, ¹⁴⁹Pm, ¹⁵¹Pm, ¹⁴⁹Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, 172Tm, 177Lu, ¹⁸⁶Re, ¹⁸⁸Re, ¹⁹¹Pt, ¹⁹⁷Hg, ¹⁹⁸Au, ¹⁹⁹Au, ²⁰¹Tl, ²⁰³Pb, ²¹¹At, ²¹²Bi, ²¹³Bi, ¹¹C, ⁷⁵Br, ⁷⁶Br, ⁷⁷Br, ⁸²Br, ¹⁸F, ¹²⁰I, ¹²³I, ¹²⁴I, ¹²⁵I, ¹³¹I, ⁸⁹Sr and ²²⁵Ac.

15. The conjugated anti-IL-5Rα compound of claim 13, wherein the radionuclide is ⁶⁴Cu.

16. The conjugated anti-IL-5Rα compound of claim 1, further comprising a chemotherapeutic agent attached thereto.

17. The conjugated anti-IL-5Rα compound of claim 16, wherein the chemotherapeutic agent is vinblastine or α-amanitin.

18. The conjugated anti-IL-5Rα compound of claim 1, for detecting bladder cancer.

19. The conjugated anti-IL-5Rα compound of claim 18, for detecting bladder cancer by PET imaging.

20. The conjugated anti-IL-5Rα compound of claim 1, for treating bladder cancer.

21. The conjugated anti-IL-5Rα compound of claim 18, wherein said bladder cancer is muscle invasive bladder cancer (MIBC).

22-25. (canceled)

26. A method of detecting and/or treating bladder cancer in a subject, comprising the step of administering to said subject the conjugated anti-IL-5Rα compound of claim 1.

27-32. (canceled)
